(19) United States
(12) Patent Application Publication
(10) Pub. No.: US 2004/0096417 A1
(43) Pub. Date: May 20, 2004

(54) Moringa esters and cosmetic and pharmaceutical preparations and methods of manufacture thereof

(76) Inventors: Robert Kleiman, Sunlakes, AZ (US); Sambasivarao Koritala, Tempe, AZ (US); John C. Hill, Mesa, AZ (US); James H. Brown, Scottsdale, AZ (US)

Correspondence Address: Michael W. Goltry, PARSONS & GOLTRY, Suite 260, 340 East Palm Lane, Phoenix, AZ, 85004, US

(21) Appl. No.: 10/294,267

(22) Filed: Nov. 14, 2002

(51) Int. Cl. A61K 35/78; A61K 7/06

(52) U.S. Cl. 424/74; 424/776

(57) ABSTRACT

Randomization of moringa oil produces moringa esters, which are useful in cosmetic and pharmaceutical preparations providing enhanced stability, improved shelf life, and enhanced slip and coverage characteristics. Exemplary randomization processes include randomizing a quantity of moringa oil with a quantity of an alkaline catalyst, randomizing a quantity of moringa oil with a quantity of a partially hydrogenated moringa oil, randomizing a quantity of moringa oil with a quantity of a completely hydrogenated moringa oil, and randomizing a quantity of moringa oil with quantities of partially hydrogenated and completely hydrogenated moringa oil.

### FIELD OF THE INVENTION

This invention relates to cosmetic and pharmaceutical preparations and to methods for enhancing the physical characteristics of cosmetic and pharmaceutical preparations.

### BACKGROUND OF THE INVENTION

It is essential that cosmetic and pharmaceutical preparations such as lipsticks, lotions, pigmented foundations, mascaras, leave-in hair preparations such as conditioners and the like, sunscreen preparations, medicated ointments, scalp treatments, acne treatments, cuticle and nail care formulations, lip protectants and hair care products exhibit exemplary slip and coverage characteristics. The provision of exemplary slip is important for ensuring easy and efficient application, and exemplary spread attributes and coverage. It is well understood among devoted artisans that stability and long shelf life of the above-named and other similar cosmetic and pharmaceutical preparations are equally important aspects. Although skilled artisans have devoted and continue to devote considerable time and effort toward beneficial additives for cosmetic preparations and to enhancing the physical characteristics of cosmetic preparations, many existing additives and associated cosmetic preparations are either expensive, difficult to manufacture and/or ineffective or not entirely effective. Given these and other deficiencies in the art, the need for certain new and useful improvements is evident.

### SUMMARY OF THE INVENTION

The above problems and others are at least partially solved and the above purposes and others realized in exemplary embodiments including improved compositions and additives for cosmetic preparations and to associated methods of manufacture thereof, in addition to associated cosmetic preparations and methods of manufacture thereof. In one exemplary embodiment, the invention is randomized moringa oil, namely, a moringa ester. This randomized moringa oil has a cloud point of 29° C. to 8° C. It is to be understood that at 100% randomization, the cloud point is 29° C., and at zero percent randomization, the cloud point is 8° C. The invention also contemplates a useful preparation (such as a cosmetic preparation or a pharmaceutical preparation) and an amount of the randomized moringa oil incorporated into the useful preparation. In a related embodiment, the invention contemplates a useful preparation (such as a cosmetic preparation or a pharmaceutical preparation) having a shelf life and an amount of the randomized moringa oil incorporated into the useful preparation, wherein the amount is sufficient to increase the shelf life. In another related embodiment, the invention contemplates a useful preparation (such as a cosmetic preparation or a pharmaceutical preparation) having a slip characteristic and an amount of the randomized moringa oil incorporated into the useful preparation, wherein the amount is sufficient to enhance the slip characteristic. In yet another related embodiment, the invention contemplates a useful preparation (such as a cosmetic preparation or a pharmaceutical preparation) having a coverage characteristic and an amount of the randomized moringa oil incorporated into the useful preparation, wherein the amount is sufficient to enhance the coverage characteristic.

In another preferred embodiment, the invention consists of a moringa ester produced from a randomization of a quantity of moringa oil and a quantity of an alkaline catalyst, such as a sodium methoxide or the like. The instant moringa ester has a cloud point of 29° C. to 8° C. The invention also contemplates a useful preparation (such as a cosmetic preparation or a pharmaceutical preparation) and an amount of the moringa ester incorporated into the useful preparation. In a related embodiment, the invention contemplates a useful (such as a cosmetic preparation or a pharmaceutical preparation) preparation having a shelf life and an amount of the moringa ester incorporated into the useful preparation, wherein the amount is sufficient to increase the shelf life. In another related embodiment, the invention contemplates a useful preparation (such as a cosmetic preparation or a pharmaceutical preparation) having a slip characteristic and an amount of the moringa ester incorporated into the useful preparation, wherein the amount is sufficient to enhance the slip characteristic. In yet another related embodiment, the invention contemplates a useful preparation (such as a cosmetic preparation or a pharmaceutical preparation) having a coverage characteristic and an amount of the moringa ester incorporated into the useful preparation, wherein the amount is sufficient to enhance the coverage characteristic.

In yet another preferred embodiment, the invention consists of a moringa ester produced from a randomization of a quantity of moringa oil and a quantity of a partially hydrogenated moringa oil. The instant moringa ester embodiment has a cloud point of 62° C. to 8° C., in which a cloud point of 62° C. represents completely hydrogenated moringa oil and 8° C. represents unrandomized unsaturated moringa oil. The invention also contemplates a useful preparation (such as a cosmetic preparation or a pharmaceutical preparation) and an amount of the moringa ester incorporated into the useful preparation. In a related embodiment, the invention contemplates a useful preparation (such as a cosmetic preparation or a pharmaceutical preparation) having a shelf life and an amount of the moringa ester incorporated into the useful preparation, wherein the amount is sufficient to increase the shelf life. In another related embodiment, the invention contemplates a useful preparation (such as a cosmetic preparation or a pharmaceutical preparation) having a slip characteristic and an amount of the moringa ester incorporated into the useful preparation, wherein the amount is sufficient to enhance the slip characteristic. In yet another related embodiment, the invention contemplates a useful preparation (such as a cosmetic preparation or a pharmaceutical preparation) having a coverage characteristic and an amount of the moringa ester incorporated into the useful preparation, wherein the amount is sufficient to enhance the coverage characteristic.

In a further preferred embodiment, the invention consists of a moringa ester produced from a randomization of a quantity of moringa oil and a quantity of a completely hydrogenated moringa oil. The instant moringa ester has a cloud point of 62° C. to 8° C., in which a cloud point of 62° C. represents completely hydrogenated moringa oil and 8° C. represents unrandomized unsaturated moringa oil. The invention also contemplates a useful preparation (such as a cosmetic preparation or a pharmaceutical preparation) and an amount of the moringa ester incorporated into the useful preparation. In a related embodiment, the invention contemplates a useful preparation (such as a cosmetic preparation or a pharmaceutical preparation) having a shelf life and an amount of the moringa ester incorporated into the useful preparation, wherein the amount is sufficient to increase the shelf life. In another related embodiment, the invention contemplates a useful preparation (such as a cosmetic preparation or a pharmaceutical preparation) having a slip characteristic and an amount of the moringa ester incorporated into the useful preparation, wherein the amount is sufficient to enhance the slip characteristic. In yet another related embodiment, the invention contemplates a useful preparation (such as a cosmetic preparation or a pharmaceutical preparation) having a coverage characteristic and an amount of the moringa ester incorporated into the useful preparation, wherein the amount is sufficient to enhance the coverage characteristic.

In still a further preferred embodiment, the invention constitutes a moringa ester produced from a randomization of a quantity of moringa oil and a) a quantity of a partially hydrogenated moringa oil and b) a quantity of a completely hydrogenated moringa oil. The instant moringa ester of claim 25 having a cloud point of 62° C. to 8° C., in which a cloud point of 62° C. represents completely hydrogenated moringa oil and 8° C. represents unrandomized unsaturated moringa oil. The invention also contemplates a useful preparation (such as a cosmetic preparation or a pharmaceutical preparation) and an amount of the moringa ester incorporated into the useful preparation. In a related embodiment, the invention contemplates a useful preparation (such as a cosmetic preparation or a pharmaceutical preparation) having a shelf life and an amount of the moringa ester incorporated into the useful preparation, wherein the amount is sufficient to increase the shelf life. In another related embodiment, the invention contemplates a useful preparation (such as a cosmetic preparation or a pharmaceutical preparation) having a slip characteristic and an amount of the moringa ester incorporated into the useful preparation, wherein the amount is sufficient to enhance the slip characteristic. In yet another related embodiment, the invention contemplates a useful preparation (such as a cosmetic preparation or a pharmaceutical preparation) having a coverage characteristic and an amount of the moringa ester incorporated into the useful preparation, wherein the amount is sufficient to enhance the coverage characteristic.

The invention also contemplates a variety of method embodiments. A preferred embodiment consists of providing a quantity of moringa oil, randomizing the quantity of moringa oil with a quantity of an alkaline catalyst to form a moringa ester having physical characteristics. The instant method further includes altering at least one of the physical characteristics of the moringa ester by varying at least one of the quantity of the moringa oil and the quantity of the alkaline catalyst. The moringa ester produced by the instant method has a cloud point of 29° C. to 8° C. There is a provision of a useful preparation (such as a cosmetic preparation or a pharmaceutical preparation) incorporating an amount of the moringa ester produced by the instant method. There is also a provision of a useful preparation (such as a cosmetic preparation or a pharmaceutical preparation) incorporating approximately 15% by weight of the moringa ester produced by the instant method. There is a further provision of a useful preparation (such as a cosmetic preparation or a pharmaceutical preparation) having a shelf life and an amount of the moringa ester of the instant method incorporated into the useful preparation, wherein the amount is sufficient to increase the shelf life. There is still a further provision of a useful preparation (such as a cosmetic preparation or a pharmaceutical preparation) having a slip characteristic and an amount of the moringa ester of the instant method incorporated into the useful preparation, wherein the amount is sufficient to enhance the slip characteristic. There is yet still a further provision of a useful preparation (such as a cosmetic preparation or a pharmaceutical preparation) having a coverage characteristic and an amount of the moringa ester of the instant method incorporated into the useful preparation, wherein the amount is sufficient to enhance the coverage characteristic.

In accordance with the invention, another preferred method embodiment consists of providing a quantity of moringa oil, and randomizing the quantity of moringa oil with a quantity of a partially hydrogenated moringa oil in the presence of an alkaline catalyst to form a moringa ester having physical characteristics. The instant method further includes altering at least one of the physical characteristics of the moringa ester by varying at least one of a) the quantity of the moringa oil, b) the quantity of the partially hydrogenated moringa oil and c) the quantity of the alkaline catalyst. The moringa ester produced by the instant method has a cloud point of 62° C. to 8° C., in which a cloud point of 62° C. represents completely hydrogenated moringa oil and 8° C. represents unrandomized unsaturated moringa oil. There is a provision of a useful preparation (such as a cosmetic preparation or a pharmaceutical preparation) incorporating an amount of the moringa ester produced by the instant method. There is also a provision of a useful preparation (such as a cosmetic preparation or a pharmaceutical preparation) incorporating approximately 15% by weight of the moringa ester produced by the instant method. There is a further provision of a useful preparation (such as a cosmetic preparation or a pharmaceutical preparation) having a shelf life and an amount of the moringa ester of the instant method incorporated into the useful preparation, wherein the amount is sufficient to increase the shelf life. There is still a further provision of a useful preparation (such as a cosmetic preparation or a pharmaceutical preparation) having a slip characteristic and an amount of the moringa ester of the instant method incorporated into the useful preparation, wherein the amount is sufficient to enhance the slip characteristic. There is yet still a further provision of a useful preparation (such as a cosmetic preparation or a pharmaceutical preparation) having a coverage characteristic and an amount of the moringa ester of the instant method incorporated into the useful preparation, wherein the amount is sufficient to enhance the coverage characteristic.

In accordance with the invention, yet another preferred method embodiment consists of providing a quantity of moringa oil, and randomizing the quantity of moringa oil with a quantity of a completely hydrogenated moringa oil in the presence of an alkaline catalyst to form a moringa ester having physical characteristics. The instant method further includes altering at least one of the physical characteristics of the moringa ester by varying at least one of a) the quantity of the moringa oil, b) the quantity of the completely hydrogenated moringa oil, and c) the quantity of the alkaline catalyst. The moringa ester produced by the instant method has a cloud point of 62° C. to 8° C., in which a cloud point of 62° C. represents completely hydrogenated moringa oil and 8° C. represents unrandomized unsaturated moringa oil. There is a provision of a useful preparation (such as a cosmetic preparation or a pharmaceutical preparation) incorporating an amount of the moringa ester produced by the instant method. There is also a provision of a useful preparation (such as a cosmetic preparation or a pharmaceutical preparation) incorporating approximately 15% by weight of the moringa ester produced by the instant method. There is a further provision of a useful preparation (such as a cosmetic preparation or a pharmaceutical preparation) having a shelf life and an amount of the moringa ester of the instant method incorporated into the useful preparation, wherein the amount is sufficient to increase the shelf life. There is still a further provision of a useful preparation (such as a cosmetic preparation or a pharmaceutical preparation) having a slip characteristic and an amount of the moringa ester of the instant method incorporated into the useful preparation, wherein the amount is sufficient to enhance the slip characteristic. There is yet still a further provision of a useful preparation (such as a cosmetic preparation or a pharmaceutical preparation) having a coverage characteristic and an amount of the moringa ester of the instant method incorporated into the useful preparation, wherein the amount is sufficient to enhance the coverage characteristic.

In accordance with the invention, still another method embodiment consists of providing a quantity of moringa oil, and randomizing the quantity of moringa oil with a) a quantity of a partially hydrogenated moringa oil and b) a quantity a completely hydrogenated moringa oil in the presence of an alkaline catalyst to form a moringa ester having physical characteristics. The instant method further includes altering at least one of the physical characteristics of the moringa ester by varying at least one of a) the quantity of the moringa oil, b) the quantity of the partially hydrogenated moringa oil, c) the quantity of the completely hydrogenated moringa oil, and d) the quantity of the alkaline catalyst. The moringa ester produced by the instant method has a cloud point of 62° C. to 8° C., in which a cloud point of 62° C. represents completely hydrogenated moringa oil and 8° C. represents unrandomized unsaturated moringa oil. There is a provision of a useful preparation (such as a cosmetic preparation or a pharmaceutical preparation) incorporating an amount of the moringa ester produced by the instant method. There is also a provision of a useful preparation (such as a cosmetic preparation or a pharmaceutical preparation) incorporating approximately 15% by weight of the moringa ester produced by the instant method. There is a further provision of a useful preparation (such as a cosmetic preparation or a pharmaceutical preparation) having a shelf life and an amount of the moringa ester of the instant method incorporated into the useful preparation, wherein the amount is sufficient to increase the shelf life. There is still a further provision of a useful preparation (such as a cosmetic preparation or a pharmaceutical preparation) having a slip characteristic and an amount of the moringa ester of the instant method incorporated into the useful preparation, wherein the amount is sufficient to enhance the slip characteristic. There is yet still a further provision of a useful preparation (such as a cosmetic preparation or a pharmaceutical preparation) having a coverage characteristic and an amount of the moringa ester of the instant method incorporated into the useful preparation, wherein the amount is sufficient to enhance the coverage characteristic.

In accordance with the teachings presented in accordance with the previously described embodiments of the invention and the teachings presented throughout the ensuing specification, which are intended to be taken together, the invention also contemplates further associated embodiments.

### DETAILED DESCRIPTION OF A PREFERRED EMBODIMENT

Randomization of moringa oil produces moringa esters, which are useful in cosmetic and pharmaceutical preparations providing enhanced stability, improved shelf life, and enhanced slip and coverage characteristics. Exemplary randomization processes include randomizing a quantity of moringa oil with a quantity of an alkaline catalyst, randomizing a quantity of moringa oil with a quantity of a partially hydrogenated moringa oil in the presence of an alkaline catalyst, randomizing a quantity of moringa oil with a quantity of a completely hydrogenated moringa oil in the presence of an alkaline catalyst, and randomizing a quantity of moringa oil with quantities of partially hydrogenated and completely hydrogenated moringa oil in the presence of an alkaline catalyst.

In accordance with the principle of the invention, this specification discloses moringa esters, which are produced by randomizing/interesterifying moringa oil, not only with itself but also with different levels/amounts of partially hydrogenated moringa oil and/or completely hydrogenated moringa oil, all in the presence of an alkaline catalyst. Moringa oil is obtained from the seed of the Moringa oleifera tree, by pressing and by extraction with hexane or other suitable solvent. Moringa oil has the lowest known amount of polyunsaturated fatty acids found naturally in seed oils. When combined with antioxidant material, moringa oil is very oxidatively stable. To increase its usefulness, the invention proposes randomizing/interesterifying moringa oil to produce moringa esters.

In one embodiment, the invention constitutes providing a quantity of moringa oil and randomizing the quantity of moringa oil with a quantity of an alkaline catalyst, such as sodium methoxide, to form a moringa ester having physical characteristics. Further to this embodiment is a provision of altering at least one of the physical characteristics of the moringa ester by varying at least one of the quantity of the moringa oil and the quantity of the alkaline catalyst. The physical characteristics of the moringa ester in accordance with this and ensuing embodiments includes, among potentially other physical characteristics or attributes, melting point, cloud point, spread value, and viscosity.

In another embodiment, the invention consists of providing a quantity of moringa oil, and randomizing the quantity of moringa oil with a quantity of a partially hydrogenated moringa oil to form a moringa ester having physical characteristics. Further to this embodiment is a provision of altering at least one of the physical characteristics of the moringa ester by varying at least one of a) the quantity of the moringa oil, b) the quantity of the partially hydrogenated moringa oil, and c) the quantity of alkaline catalyst.

In still another embodiment, the invention consists of providing a quantity of moringa oil, and randomizing the quantity of moringa oil with a quantity of a completely hydrogenated moringa oil to form a moringa ester having physical characteristics. Further to this embodiment is a provision of altering at least one of the physical characteristics of the moringa ester by varying at least one of a) the quantity of the moringa oil, b) the quantity of the completely hydrogenated moringa oil and c) the quantity of alkaline catalyst.

In yet still another embodiment, the invention constitutes providing a quantity of moringa oil, and randomizing the quantity of moringa oil with a) a quantity of a partially hydrogenated moringa oil and b) a quantity a completely hydrogenated moringa oil to form a moringa ester having physical characteristics. Further to this embodiment is a provision of altering at least one of the physical characteristics of the moringa ester by varying at least one of a) the quantity of the moringa oil, b) the quantity of the partially hydrogenated moringa oil, c) the quantity of the completely hydrogenated moringa oil and d) the quantity of alkaline catalyst.

The moringa esters resulting from these reactions possess properties that are unexpected. By varying the ratio of moringa oil and hydrogenated moringa oil in the interesterifying reactions the melting points and other properties of the products can, be changed. In accordance with this disclosure, 1) Moringa Ester-15 (ME-15) is a moringa ester produced from a randomization or interesterifying of moringa oil, 2) Moringa Ester-20 (ME-20) is a moringa ester produced from a randomization or transesterification of four (4) parts moringa oil and one (1) part hydrogenated moringa oil, 3) Moringa Ester-30 (ME-30) is a moringa ester produced from a randomization or interesterifying of two-point-three (2.3) parts moringa oil and one (1) part hydrogenated moringa oil, 4) Moringa Ester-60 (ME-60) is a moringa ester produced from a randomization or interesterifying of one (1) part moringa oil and one (1) part hydrogenated moringa oil, 5) Moringa Ester-75 (ME-75) is a moringa ester produced from a randomization or interesterifying of one (1) part moringa oil and three (3) parts hydrogenated moringa oil, and 6) Moringa Ester-100 (ME-100) is a moringa ester produced from a randomization or interesterifying of completely hydrogenated moringa oil. The foregoing are examples of different moringa esters. Consistent with this disclosure, the invention contemplates other varieties of moringa esters.

Moringa Ester-15, for example, has unexpected differences when compared to unchanged moringa oil. Though both are triglyceride in nature and have the same fatty acid composition, the acyl groups of ME-15 are randomly esterified on the triglyceride molecule while the placement of fatty acyl groups of moringa oil are controlled by the plant's enzyme system. One of the consequences of randomization or interesterifying is that the cloud point of Moringa-15 is much higher than unchanged moringa oil, 29° C. to 8° C., making ME-15 semisolid at room temperature while the natural or unchanged/crude moringa oil is liquid under the same conditions. The spread values of Moringa-15 and unchanged moringa oil are also different, in which Moringa-15 has a spread value of approximately 11.3% and unchanged moringa oil has a spread value of 18.6%. In lipsticks prepared using 15% ME-15 and 15% unchanged moringa oil, respectively, the dropping point (AOCS Official Method Cc 18-80) of the lipstick using ME-15 is 2.7% greater than the dropping point of the lipstick incorporating the unchanged moringa oil. In lipsticks prepared using 15% ME-15 and 15% unchanged moringa oil, respectively, the spread characteristic of the lipstick using ME-15 is lower than the spread characteristic of the lipstick incorporating the unchanged moringa oil. The provision of ME-15, and the other moringa esters disclosed in this specification, in lipstick applications is, therefore, very useful in providing lipstick products having improved dropping point and lower spread characteristics. The lower spread provided by a lipstick incorporating 15% ME-15, the other moringa esters disclosed therein and selected combinations of the moringa esters disclosed herein, results in less lipstick “feathering” when on the lips and the higher dropping point will improve the lasting properties of such a lipstick. These properties also pertain to other stick type products including lip balms and lip protection sticks.

In accordance with the provisions of the invention, the moringa esters disclosed herein with the addition of an antioxidant such as BHA, BHT, Propyl gallate, or a tocopherol material at selected and suitable levels/amounts, whether one tocopherol or a combination of selected tocopherols and other antioxidants, exhibit surprisingly high oxidative stabilities that exceed expected values and that exceed the oxidative stabilities of corresponding jojoba esters and this aspect is seen in Graph 1, infra. The use of these highly stable moringa esters improves the shelf life of lipstick products and other color cosmetics especially where prooxidant pigments such as iron oxides are used.

[image]

The ease of application of cosmetic products is known by those skilled in the art as “slip.” The easier a product is to apply and the less force required to apply the product the greater the slip of the product. As seen in Graph 2, infra, comparisons of slip values between the novel moringa esters disclosed herein and corresponding jojoba esters show a much greater than expected slip for the moringa esters as compared to the corresponding jojoba esters. This unexpected improvement allows lipsticks and other stick cosmetics to be applied easier and is useful in other cosmetics in which exemplary slip is desired. Jojoba Ester 75 was not available for testing.

[image]

To demonstrate the difference between the slip properties of a cosmetic made with the moringa esters disclosed herein and corresponding jojoba esters, the inventors of the subject matter of this specification provided two creamy foundation formulas, one made with jojoba esters and another made with corresponding moringa esters, namely, a combination of ME-15 and ME-30 provided at 15% by weight of the product. A panel of twelve individuals, in a blind test of these foundations, 83% of a panel of twelve (12) individuals chose the moringa ester creamy foundation formula over the jojoba ester creamy foundation formula as having superior slip, less drag, and better coverage.

The moringa esters disclosed herein are successfully used, both individually and at varying weight percentages in addition to various combinations and weight percentages thereof, in many types of useful preparations, namely, cosmetic and pharmaceutical formulations/preparations. These formulation types include lipstick formulations, lotion formulations, pigmented foundation formulations, mascara formulations, leave-in hair preparations such as conditioners and the like and other hair care product formulations, scalp treatment formulations, cuticle and nail care formulations, sunscreen formulations, lip protectants and balm formulations, acne treatment formulations and medicated ointments, etc. The use of moringa esters in each of these formulation types demonstrates not only the usefulness of moringa esters but also the ability to mix moringa esters with common cosmetic and pharmaceutical ingredients including but not limited to pigments, waxes, silicones, alcohols, triglycerides, emulsifiers, anti-oxidants, medications, other “actives” and preservatives.

Moringa esters have excellent lubrication properties. In addition to the previously described properties and benefits of moringa esters, the high slip and excellent lubrication properties of moringa esters in pigmented cosmetic preparations results in a high degree of pigment dispersion. And so by exploiting the high slip properties of the moringa esters disclosed herein, the cosmetic formulator can achieve a high degree of pigment dispersion. This is demonstrated in pigmented cosmetic formulations such as lipsticks, pigmented foundations, and mascaras, lotions, sunscreen preparations, nail care formulations, lip protectants, etc. The high slip properties also result in easier application of stick products and better payout of the product, which is highly beneficial. The use of moringa esters in stick product cosmetic formulations results in enhanced slip and coverage characteristics and extended wear. The use of moringa esters in lotions enhances viscosity, and in mascara formulations contributes to extended wear and stability. The use of moringa esters to exploit their enhanced oxidative stability in pharmaceutical containing “actives” or in highly pigmented cosmetic preparations is desirable.

In accordance with the principle of the invention, examples of lipstick formulations incorporating moringa esters and associated manufacturing techniques are disclosed in Table I, infra.

TABLE I
Lipstick Evaluation Formulas

| Phase | Trade Name | ME-15 | ME-20 | ME-30 | ME-60 |
|---|---|---|---|---|---|
| A. | Castor Oil (CasChem) | 41.0 | 41.0 | 41.0 | 41.0 |
| | Moringa Esters 15 | 15.0 | 0 | 0 | 0 |
| | Moringa Esters 20 | 0 | 15.0 | 0 | 0 |
| | Moringa Esters 30 | 0 | 0 | 15.0 | 0 |
| | Moringa Esters 60 | 0 | 0 | 0 | 15.0 |
| | Moringa Esters 75 | 1.0 | 1.0 | 1.0 | 1.0 |
| | Carnauba Wax #1 Yellow (Strahl & Pitsch) | 4.0 | 4.0 | 4.0 | 4.0 |
| | Candelilla Wax (Strahl & Pitsch) | 5.0 | 5.0 | 5.0 | 5.0 |
| | Beeswax (Strahl & Pitsch) | 3.5 | 3.5 | 3.5 | 3.5 |
| | Microcrystalline Wax (Strahl & Pitsch) | 4.0 | 4.0 | 4.0 | 4.0 |
| | Propylparaben | 0.1 | 0.1 | 0.1 | 0.1 |
| | Cabosil M-5 (Cabot) | 0.2 | 0.2 | 0.2 | 0.2 |
| B. | Castor Oil (CasChem) | 13.0 | 13.0 | 13.0 | 13.0 |
| | Arlacel P-100 (ICI) | 0.5 | 0.5 | 0.5 | 0.5 |
| | Titanium Dioxide (Warner- | 5.5 | 5.5 | 5.5 | 5.5 |
| | Jenkinson) | | | | |
| | Pur Oxy Red - 3511 (Hilton-Davis) | 4.0 | 4.0 | 4.0 | 4.0 |
| | D&C Red #30, Talc Lake - 3130 | 2.5 | 2.5 | 2.5 | 2.5 |
| | (Hilton Davis) | | | | |
| | FD&C Blue #1, Aluminum Lake | 0.1 | 0.1 | 0.1 | 0.1 |
| | (Warner Jenkinson) | | | | |
| | Black Iron Oxide - 3070 (Hilton-Davis) | 0.5 | 0.5 | 0.5 | 0.5 |
| C. | Covi-Ox T-70 (B&D Nutritional) | 0.1 | 0.1 | 0.1 | 0.1 |
| | Total | 100.0 | 100.0 | 100.0 | 100.0 |

Mixing/manufacturing procedure

| | |
|---|---|
| 1. | Combine ingredients of Phase A and heat to 85° C. with moderate |
| | agitation. |
| 2. | Combine ingredients of Phase B and pass two times through a |
| | three-roll mill. |
| 3. | Add Phase B to Phase A with propeller agitation, forming a |
| | batch mixture. |
| 4. | Cool the batch mixture to 75° C. |
| 5. | Add Phase C to the batch mixture and mix with propeller |
| | agitation, forming a lipstick formulation. |
| 6. | Fill lipstick molds with the lipstick formulation. |

In accordance with the invention, Formula I, infra, is a cream makeup foundation formulation with moringa esters. The moringa-derived moringa esters in this cream makeup foundation formulation provide the formulation with improved the skin feel, improved application and coverage properties, and improved “playtime.”

Formula I. Makeup Foundation with Moringa Esters: Long-
Wearing Creamy Moringa Esters Foundation

| | | | | % wt./ |
|---|---|---|---|---|
| Phase | Trade Name | INCI Name | Supplier | wt. |
| A. | Deionized Water | Water | — | 44.05 |
| | CMC (7MF) | Cellulose Gum | Aqualon | 0.30 |
| | Veegum | Magnesium Aluminum | R. T. Vanderbilt | 0.80 |
| | | Silicate | | |
| | Propylene Glycol | Propylene Glycol | Dow | 5.00 |
| B. | Propylene Glycol | Propylene Glycol | Dow | 5.00 |
| | Floraesters K-20W | Hydrolyzed Jojoba Esters | Floratech | 10.00 |
| | Jojoba | (and) Water | | |
| C. | Supra H | Talc | Luzenac | 3.00 |
| | Kowet | Titanium Dioxide | Warner-Jenkinson | 11.00 |
| | Pur Oxy Red B.C. 34- | Iron Oxides | Noveon | 0.20 |
| | 3080 | | | |
| | Pur Oxy Yellow B.C. | Iron Oxides | Noveon | 1.14 |
| | 34-3170 | | | |
| | Pur Oxy Red B.C. 34- | Iron Oxides | Noveon | 0.24 |
| | 3551 | | | |
| | Pur Oxy Black B.C. | Iron Oxides | Noveon | 0.24 |
| | 34-3068 | | | |
| | Triethanolamine 99% | Triethanolamine | Dow | 0.40 |
| D. | Standapol SH-135 | Disodium Oleamido PEG-2 | Cognis | 0.13 |
| | | Sulfosuccinate | | |
| E. | Moringa Esters 15 | Moringa Esters | Floratech | 8.00 |
| | Moringa Esters 30 | Moringa Esters | Floratech | 3.00 |
| | Vitamin E Acetate CG | Tocopheryl Acetate | Roche | 0.20 |
| | Lanette 16 | Cetyl Alcohol | Cognis | 0.50 |
| | Pristerene 4911 | Stearic Acid | Uniqema | 2.40 |
| | Lexemul 561 | Glyceryl Stearate (and) | Inolex | 0.60 |
| | | PEG-100 Stearate | | |
| | SF 96-350 Silicone | Dimethicone | GE Silicones | 0.20 |
| | Fluid | | | |
| | SF 1202 Silicone | Cyclopentasiloxane | GE Silicones | 3.00 |
| | Fluid | | | |
| F. | Gemaben II | Propylene Glycol (and) | ISP Sutton | 0.60 |
| | | Diazolidinyl Urea (and) | | |
| | | Methylparaben (and) | | |
| | | Propylparaben | | |
| | | TOTAL: | | 100.00 |

Mixing/manufacturing procedure:

| | |
|---|---|
| 1. | Phase A-Disperse CMC and Veegum in propylene glycol, |
| | forming a gum dispersion. Heat deionized water to 75° C. |
| | Add the gum dispersion to the deionized water and mix for |
| | thirty (30) minutes or until the gum material of the gum |
| | dispersion are hydrated. |
| 2. | Phase B-Dissolve K-20W of Phase into propylene glycol |
| | until uniform. Add Phase B to Phase A at 75° C. with |
| | propeller agitation, forming Phase AB. |
| 3. | Add constituents of Phase C to Phase AB at 75° C. with |
| | high-speed homomixer agitation. Mix for 30 minutes, |
| | forming Phase ABC. |
| 4. | Add Phase D to Phase ABC with moderate homomixer |
| | agitation. Mix 10 minutes and hold at 80° C., forming |
| | Phase ABCD. |
| 5. | Combine Phase E ingredients and heat to 80° C. with |
| | propeller agitation. Heat Phase E at 80° C., and add Phase |
| | E slowly to Phase ABCD with moderate propeller agitation. |
| | Mix 20 minutes and force cool with propeller agitation to |
| | 75° C., forming Phase ABCDE. |
| 6. | Add Phase F to Phase ABCDE at 40-45° C. and cool to room |
| | temperature with moderate propeller agitation. |
| 7. | Adjust pH as necessary to pH 7.4 ± 0.1. |

By blending moringa esters into the oil phase of a formulation, it is possible to make both oil-in-water and water-in-oil emulsions, namely, lotions. Several formulas have been made, which demonstrate compatibility of moringa esters with popular emulsifiers when making lotions. Depending on the moringa ester type used or the combination of moringa ester types used, formulas can be tailored for high or low viscosity, varying degrees of richness and very smooth skin feel. When organic or inorganic chemical UVA and UVB absorbers are added to such formulas, very effective sunscreen lotions have been produced. Formula II, infra, is an example of a lotion of an oil-in-water emulsion type created with moringa esters.

### Formula II. Lotion containing Moringa Esters: Moringa Esters Lotion for Dry Skin

Moringa esters create a natural, stable, oil-free emollient system in the ensuing embodiment is a soothing lotion for dry skin. Paired combinations of ME-20, ME-30 and ME-60 can be used to adjust the viscosity of the final lotion.

| | | | | Formula 1 | Formula 2 |
|---|---|---|---|---|---|
| Phase | Trade Name | INCI Name | Supplier | % wt./wt. | % wt./wt. |
| A. | Moringa Esters 20 | Moringa Esters | Floratech | 5.00 | 0.00 |
| | Moringa Esters 30 | Moringa Esters | Floratech | 5.00 | 5.00 |
| | Moringa Esters 60 | Moringa Esters | Floratech | 0.00 | 5.00 |
| | Ethyl Moringate | Ethyl Moringate | Floratech | 5.00 | 5.00 |
| | Brij 72 | Steareth-2 | Uniqema | 3.00 | 3.00 |
| | Brij 721 | Steareth-21 | Uniqema | 1.00 | 1.00 |
| | Lanette 18 | Stearyl Alcohol | Cognis | 4.00 | 4.00 |
| | Preservative | — | | Q.S. | Q.S. |
| B. | Deionized Water | Water | | Q.S. | Q.S. |
| | Glycerine 99% | Glycerin | Dow | 3.00 | 3.00 |
| | Preservative | — | | Q.S. | Q.S. |
| C. | Deionized Water | Water | | Q.S. | Q.S. |
| | | TOTAL: | | 100.00 | 100.00 |

Mixing/manufacturing procedure:

| | |
|---|---|
| 1. | Heat Phase A to 75° C. with agitation. |
| 2. | Heat Phase B to 75° C. with agitation. Note: In accordance |
| | with a preferred embodiment, use 1/3 the Q.S. water in |
| | Phase B, and 2/3 in Phase C. |
| 3. | Add Phase A slowly to Phase B with homomixer agitation to |
| | create fine particle size emulsion droplets. Force cool |
| | to 50° C. with agitation, forming Phase AB. |
| 4. | Add Phase C to Phase AB and mix with propeller agitation |
| | to room temperature. |
| | Moringa Esters enhance stick strength in stick type |

products with wax base compositions. As an example, stronger stick strengths are achieved by using Moringa esters-60 and 75 at selected amounts and ratios, and lower stick strengths are achieved by using Moringa esters-30 and/or 20 at selected amounts and ratios. Any desired combination of moringa esters in a wax base product can be employed for varying the stick strengths of such stick products, including lipsticks and lip balms. Formula III, infra, is an example of a lip balm formulation using moringa esters. In addition, this methodology was also used to produce long lasting mascara formulations.

### Formula III. Moringa Esters in a Stick Formula Natural Moringa Esters Lip Protectant

Moringa esters form a protective, emollient moisture barrier on the lips that helps keep them soft and supple when exposed to the elements (wind, sun, cold and dry heat). Each of the natural botanical emollients in the ensuing formula exhibits excellent oxidative stability.

| Phase | Trade Name | INCI Name | Supplier | % wt./wt. |
|---|---|---|---|---|
| A. | Castor Oil | Ricinus Communis | CasChem | Q.S. |
| | | (Castor) Seed Oil | | |
| | Floramac Hawaiian | Macadamia Integrifolia | Floratech | 2.10 |
| | Macadamia Nut Oil | Seed Oil | | |
| | Moringa Esters 30 | Moringa Esters | Floratech | 13.00 |
| | Moringa Esters 60 | Moringa Esters | Floratech | 6.50 |
| | Moringa Esters 75 | Moringa Esters | Floratech | 1.00 |
| | Carnauba Wax #1 Yellow | Copernicia Cerifera | Strahl & | 5.10 |
| | SP 63 | (Carnauba) Wax | Pitsch | |
| | Candelilla Wax SP 75 | Euphorbia Cerifera | Strahl & | 5.10 |
| | | (Candelilla) Wax | Pitsch | |
| | Yellow Beeswax SP 6P | Beeswax | Strahl & | 3.00 |
| | | | Pitsch | |
| | Castorwax-NF | Hydrogenated Castor | CasChem | 1.00 |
| | | Oil | | |
| | NatureChem CR | Cetyl Ricinoleate | CasChem | 2.00 |
| | Preservative | — | | Q.S. |
| B. | Eusolex T-45D | Isononyl Isonononate | Rona | 1.215 |
| | | (and) Titanium | | |
| | | Dioxide (and) Alumina | | |
| | | (and) Simethicone | | |
| | | (and) Polyglyceryl-6 | | |
| | | Ricinoleate | | |
| C. | β-Carotene 30% | Beta-Carotene | Roche | 0.003 |
| | Parsol MCX | Ethylhexyl | Roche | 5.00 |
| | | Methoxycinnamate | | |
| | Covi-Ox T-70 | Tocopherol | Cognis | 0.50 |
| | Orange Oil Natural 10- | Flavor | SunPure, | 2.00 |
| | Fold | | Ltd.. | |
| | | TOTAL: | | 100.00 |

Mixing/manufacturing procedure:

| | |
|---|---|
| 1. | Combine ingredients of Phase A and heat to 85° C. with |
| | moderate agitation. |
| 2. | Add Phase B to Phase A and mix with propeller agitation, |
| | forming Phase AB. |
| 3. | Cool Phase AB to 75° C., add Phase C to Phase AB and mix |
| | with propeller agitation. |

In summary, through research with many various types of cosmetic and pharmaceutical products, it has been shown that the moringa esters disclosed herein are compatible with most types of cosmetic and pharmaceutical raw materials. This list of raw materials includes, but is not limited to, waxes, pigments, emollients, solvents, pharmaceutical actives, silicones, alcohols, squalene, triglycerides, antioxidants, preservatives, emulsifiers, humectants and botanicals.

The present invention is described above with reference to preferred embodiments. However, those skilled in the art will recognize that changes and modifications may be made in the described embodiments without departing from the nature and scope of the present invention. Various changes and modifications to the embodiments herein chosen for purposes of illustration will readily occur to those skilled in the art. To the extent that such modifications and variations do not depart from the spirit of the invention, they are intended to be included within the scope thereof.

Having fully described the invention in such clear and concise terms as to enable those skilled in the art to understand and practice the same, the invention claimed is:

1. A randomized moringa oil.

2. The randomized moringa oil of claim 1 having a cloud point of 29° C. to 8° C.

3. A cosmetic or pharmaceutical preparation and an amount of the randomized moringa oil of claim 1 incorporated into the cosmetic or pharmaceutical preparation.

4. A cosmetic or pharmaceutical preparation having a shelf life and an amount of the randomized moringa oil of claim 1 incorporated into the cosmetic or pharmaceutical preparation, wherein the amount is sufficient to increase the shelf life.

5. A cosmetic or pharmaceutical preparation having a slip characteristic and an amount of the randomized moringa oil of claim 1 incorporated into the cosmetic or pharmaceutical preparation, wherein the amount is sufficient to enhance the slip characteristic.

6. A cosmetic or pharmaceutical preparation having a coverage characteristic and an amount of the randomized moringa oil of claim 1 incorporated into the cosmetic preparation, wherein the amount is sufficient to enhance the coverage characteristic.

7. A moringa ester produced from a randomization of a quantity of moringa oil and a quantity of an alkaline catalyst.

8. The moringa ester of claim 7 having a cloud point of 29° C. to 8° C.

9. A cosmetic or pharmaceutical preparation and an amount of the moringa ester of claim 7 incorporated into the cosmetic or pharmaceutical preparation.

10. A cosmetic or pharmaceutical preparation having a shelf life and an amount of the moringa ester of claim 7 incorporated into the cosmetic or pharmaceutical preparation, wherein the amount is sufficient to increase the shelf life.

11. A cosmetic or pharmaceutical preparation having a slip characteristic and an amount of the moringa ester of claim 7 incorporated into the cosmetic or pharmaceutical preparation, wherein the amount is sufficient to enhance the slip characteristic.

12. A cosmetic or pharmaceutical preparation having a coverage characteristic and an amount of the randomized moringa oil of claim 7 incorporated into the cosmetic or pharmaceutical preparation, wherein the amount is sufficient to enhance the coverage characteristic.

13. A moringa ester produced from a randomization of a quantity of moringa oil and a quantity of a partially hydrogenated moringa oil.

14. The moringa ester of claim 13 having a cloud point of 62° C. to 8° C.

15. A cosmetic or pharmaceutical preparation and an amount of the moringa ester of claim 13 incorporated into the cosmetic or pharmaceutical preparation.

16. A cosmetic or pharmaceutical preparation having a shelf life and an amount of the moringa ester of claim 13 incorporated into the cosmetic or pharmaceutical preparation, wherein the amount is sufficient to increase the shelf life.

17. A cosmetic or pharmaceutical preparation having a slip characteristic and an amount of the moringa ester of claim 13 incorporated into the cosmetic or pharmaceutical preparation, wherein the amount is sufficient to enhance the slip characteristic.

18. A cosmetic or pharmaceutical preparation having a coverage characteristic and an amount of the randomized moringa oil of claim 13 incorporated into the cosmetic or pharmaceutical preparation, wherein the amount is sufficient to enhance the coverage characteristic.

19. A moringa ester produced from a randomization of a quantity of moringa oil and a quantity of a completely hydrogenated moringa oil.

20. The moringa ester of claim 19 having a cloud point of 62° C. to 8° C.

21. A cosmetic or pharmaceutical preparation and an amount of the moringa ester of claim 19 incorporated into the cosmetic or pharmaceutical preparation.

22. A cosmetic or pharmaceutical preparation having a shelf life and an amount of the moringa ester of claim 19 incorporated into the cosmetic or pharmaceutical preparation, wherein the amount is sufficient to increase the shelf life.

23. A cosmetic or pharmaceutical preparation having a slip characteristic and an amount of the moringa ester of claim 19 incorporated into the cosmetic or pharmaceutical preparation, wherein the amount is sufficient to enhance the slip characteristic.

24. A cosmetic or pharmaceutical preparation having a coverage characteristic and an amount of the randomized moringa oil of claim 19 incorporated into the cosmetic or pharmaceutical preparation, wherein the amount is sufficient to enhance the coverage characteristic.

25. A moringa ester produced from a randomization of a quantity of moringa oil and a) a quantity of a partially hydrogenated moringa oil and b) a quantity of a completely hydrogenated moringa oil.

26. The moringa ester of claim 25 having a cloud point of 62° C. to 8° C.

27. A cosmetic or pharmaceutical preparation and an amount of the moringa ester of claim 25 incorporated into the cosmetic or pharmaceutical preparation.

28. A cosmetic or pharmaceutical preparation having a shelf life and an amount of the moringa ester of claim 25 incorporated into the cosmetic or pharmaceutical preparation, wherein the amount is sufficient to increase the shelf life.

29. A cosmetic or pharmaceutical preparation having a slip characteristic and an amount of the moringa ester of claim 25 incorporated into the cosmetic or pharmaceutical preparation, wherein the amount is sufficient to enhance the slip characteristic.

30. A cosmetic or pharmaceutical preparation having a coverage characteristic and an amount of the randomized moringa oil of claim 25 incorporated into the cosmetic or pharmaceutical preparation, wherein the amount is sufficient to enhance the coverage characteristic.

31. A method comprising steps of:
providing a quantity of moringa oil; and
randomizing the quantity of moringa oil with a quantity of an alkaline catalyst to form a moringa ester having physical characteristics.

32. The method of claim 31, further including altering at least one of the physical characteristics of the moringa ester by varying at least one of the quantity of the moringa oil and the quantity of the alkaline catalyst.

33. The moringa ester produced by the method of claim 31 having a cloud point of 29° C. to 8° C.

34. A cosmetic or pharmaceutical preparation incorporating an amount of the moringa ester produced by the method of claim 31.

35. A cosmetic or pharmaceutical preparation incorporating approximately 15% by weight of the moringa ester produced by the method of claim 31.

36. A cosmetic or pharmaceutical preparation having a shelf life and an amount of the moringa ester of claim 31 incorporated into the cosmetic or pharmaceutical preparation, wherein the amount is sufficient to increase the shelf life.

37. A cosmetic or pharmaceutical preparation having a slip characteristic and an amount of the moringa ester of claim 31 incorporated into the cosmetic or pharmaceutical preparation, wherein the amount is sufficient to enhance the slip characteristic.

38. A cosmetic or pharmaceutical preparation having a coverage characteristic and an amount of the moringa ester of claim 31 incorporated into the cosmetic or pharmaceutical preparation, wherein the amount is sufficient to enhance the coverage characteristic.

39. A method comprising steps of:
providing a quantity of moringa oil; and
randomizing the quantity of moringa oil with a quantity of a partially hydrogenated moringa oil in the presence of an alkaline catalyst to form a moringa ester having physical characteristics.

40. The method of claim 33, further including altering at least one of the physical characteristics of the moringa ester by varying at least one of a) the quantity of the moringa oil, b) the quantity of the partially hydrogenated moringa oil and c) the quantity of the alkaline catalyst.

41. The moringa ester produced by the method of claim 39 having a cloud point of 62° C. to 8° C.

42. A cosmetic or pharmaceutical preparation incorporating an amount of the moringa ester produced by the method of claim 39.

43. A cosmetic or pharmaceutical preparation incorporating approximately 15% by weight of the moringa ester produced by the method of claim 39.

44. A cosmetic or pharmaceutical preparation having a shelf life and an amount of the moringa ester of claim 39 incorporated into the cosmetic or pharmaceutical preparation, wherein the amount is sufficient to increase the shelf life.

45. A cosmetic or pharmaceutical preparation having a slip characteristic and an amount of the moringa ester of claim 39 incorporated into the cosmetic or pharmaceutical preparation, wherein the amount is sufficient to enhance the slip characteristic.

46. A cosmetic or pharmaceutical preparation having a coverage characteristic and an amount of the moringa ester of claim 39 incorporated into the cosmetic or pharmaceutical preparation, wherein the amount is sufficient to enhance the coverage characteristic.

47. A method comprising steps of:
providing a quantity of moringa oil; and
randomizing the quantity of moringa oil with a quantity of a completely hydrogenated moringa oil in the presence of an alkaline catalyst to form a moringa ester having physical characteristics.

48. The method of claim 47, further including altering at least one of the physical characteristics of the moringa ester by varying at least one of a) the quantity of the moringa oil, b) the quantity of the partially hydrogenated moringa oil and c) the quantity of the alkaline catalyst.

49. The moringa ester produced by the method of claim 47 having a cloud point of 62° C. to 8° C.

50. A cosmetic or pharmaceutical preparation incorporating an amount of the moringa ester produced by the method of claim 47.

51. A cosmetic or pharmaceutical preparation incorporating approximately 15% by weight of the moringa ester produced by the method of claim 47.

52. A cosmetic or pharmaceutical preparation having a shelf life and an amount of the moringa ester of claim 47 incorporated into the cosmetic or pharmaceutical preparation, wherein the amount is sufficient to increase the shelf life.

53. A cosmetic or pharmaceutical preparation having a slip characteristic and an amount of the moringa ester of claim 47 incorporated into the cosmetic or pharmaceutical preparation, wherein the amount is sufficient to enhance the slip characteristic.

54. A cosmetic or pharmaceutical preparation having a coverage characteristic and an amount of the moringa ester of claim 47 incorporated into the cosmetic or pharmaceutical preparation, wherein the amount is sufficient to enhance the coverage characteristic.

55. A method comprising steps of:
providing a quantity of moringa oil; and
randomizing the quantity of moringa oil with a quantity of a partially hydrogenated moringa oil and b) a quantity a completely hydrogenated moringa oil in the presence of an alkaline catalyst to form a moringa ester having physical characteristics.

56. The method of claim 55, further comprising altering at least one of the physical characteristics of the moringa ester by varying at least one of a) the quantity of the moringa oil, b) the quantity of the partially hydrogenated moringa oil, c) the quantity of the completely hydrogenated moringa oil, and d) the quantity of the alkaline catalyst.

57. The moringa ester produced by the method of claim 55 having a cloud point of 62° C. to 8° C.

58. A cosmetic or pharmaceutical preparation incorporating an amount of the moringa ester produced by the method of claim 55.

59. A cosmetic or pharmaceutical preparation incorporating approximately 15% by weight of the moringa ester produced by the method of claim 55.

60. A cosmetic or pharmaceutical preparation having a shelf life and an amount of the moringa ester of claim 55 incorporated into the cosmetic or pharmaceutical preparation, wherein the amount is sufficient to increase the shelf life.

61. A cosmetic or pharmaceutical preparation having a slip characteristic and an amount of the moringa ester of claim 55 incorporated into the cosmetic or pharmaceutical preparation, wherein the amount is sufficient to enhance the slip characteristic.

62. A cosmetic or pharmaceutical preparation having a coverage characteristic and an amount of the moringa ester of claim 55 incorporated into the cosmetic or pharmaceutical preparation, wherein the amount is sufficient to enhance the coverage characteristic.
